(19) United States
(12) Patent Application Publication
(10) Pub. No.: US 2009/0202616 A1
(43) Pub. Date: Aug. 13, 2009

(54) Composite, Method of Producing the Composite and Uses of the Same

(75) Inventors: Ee Jay Chong, Singapore (SG); Chwee Teck Lim, Singapore (SG); Seeram Ramakrishna, Singapore (SG); Toan Thang Phan, Singapore (SG); Ivor Jiun Lim, Astridville (SG)

Correspondence Address: QUARLES & BRADY LLP, 33 E. MAIN ST, SUITE 900, P.O BOX 2113, MADISON, WI, 53701-2113, US

(73) Assignee: NATIONAL UNIVERSITY OF SINGAPORE, Singapore (SG)

(21) Appl. No.: 11/664,164

(22) PCT Filed: Sep. 29, 2005
(86) PCT No.: PCT/SG05/00323

§ 371 Date: Mar. 18, 2009

Related U.S. Application Data
(60) Provisional application No. 60/614,431, filed on Sep. 29, 2004.

(51) Int. Cl. A61K 9/00; C12N 5/08; B29C 47/04; A61P 17/02

(52) U.S. Cl. 424/447; 435/371; 264/465

(57) ABSTRACT

The invention relates to a composite comprising a semi-permeable barrier layer that is permeable to oxygen and impermeable to moisture; and a scaffold fiber layer formed by electrospinning fibers on one side of the barrier layer.

### TECHNICAL FIELD

The present invention generally relates to a composite, a method of making that composite and uses of the same. The present invention also relates to a kit comprising the composite.

### BACKGROUND

According to worldwide statistics, an astonishing annual amount of US$5 billion is spent on burn wound care. In United Kingdom (UK), about £600 million are spent every year for the treatment of chronic leg ulceration. In the United States (USA), the estimated expenditure on wound care products is about US$9 billion. The average cost of wound healing ranges from about US$27,000 for a pressure ulcer to about US$36,000 for a diabetic wound. In Singapore, the estimated amount spent on chronic wounds is about US$180 million per year.

Human skin is a relatively soft tissue and yet must be able to withstand large shear stresses. Heat, chemicals, electricity, ultraviolet, or nuclear energy can cause injuries to the skin resulting in various degrees of skin damage. The least degree of injury to the skin occurs at the epithelium, which is the uppermost layer of the skin. In such cases, wounded epithelium generally is healed via re-epithelialization and does not require any skin grafting. However, more serious damage to the skin may lead to partial or complete damage to both the dermal and subdermal tissues.[4] For such cases, the body is unable to heal itself. Since skin forms a protective barrier around the human body, damage to the dermis poses several immediate threats such as rapid, severe dehydration and various forms of infection.

In order to prevent such threats from happening, there are a few suggested measures that can be readily applied. The first is that of an autograft, where a section of skin is removed from another part of the body and is subsequently grafted onto the wound. However, the drawback is that removal of the dermis and epidermis is a serious operation and if the burns are widespread, there may be insufficient healthy skin available to graft onto all the burnt areas. Deep scarring will be prominent at the area of excision.

The second option would be the use of permanent skin replacement products, which are now widely available in the market for example Integra (Integra Life Sciences, Plainsboro, N.J., USA). Integra is made up of a bi-laminate membrane consisting of a bovine collagen-based dermal analogue and a temporary epidermal substitute layer of silicone. The dermal replacement layer of Integra consists of a porous matrix of fibers of bovine type I collagen that is crosslinked with chondrotin-6-sulfate, and glycosaminoglycan (GAG) extracted from shark cartilage.

Integra is placed on the excised wound until the formation of neodermis. After the neodermis has been formed, the silicone layer is removed and a thin epidermal autograft of around 0.005 inch is applied. During the period between placement and epidermal autograft, the Integra graft must be protected from mechanical dislodgement and observed daily for signs and symptoms of infection. The disadvantage of Integra application is the difficulty in producing ultra-thin epidermal autograft. In addition, the small size of the Integra and high cost involved in production makes it unaffordable for the general public.

Another commercially available wound dressing material is Dermagraft® (Advanced Tissue Sciences, La Jolla, Calif., USA) that is a cryopreserved human allograft fibroblast-derived dermal substitute comprising of fibroblasts, ECM and a bioabsorbable scaffold.

The disadvantage of Dermagraft® is that it cannot be used in ulcers that have signs of clinical infection or sinus tracts. Also utility of Dermagraft® in wounds that extend to the tendon, muscle, joint capsules or bone has not been tested.

Another commercially available wound dressing material is TransCyte® (Advanced Tissue Sciences, La Jolla, Calif., USA) that consists of a polymer membrane and neonatal human fibroblast cells cultured under aseptic conditions in vitro on a porcine collagen coated nylon mesh. It acts as a temporary wound covering for surgically excised full-thickness and partial-thickness wounds, to protect the wound from environmental insults. In addition, the membrane is semi-permeable, thus allowing fluid and gaseous exchange.

The disadvantage of TransCyte® is that it cannot be applied to patients who are sensitive to porcine dermal collagen. TransCyte® may also contain small traces of animal proteins due to exposure in the manufacturing process, and similarly in the pre-coating of the nylon mesh with porcine dermal collagen. The traces of animal proteins may be source of pirons. There are also some ethical issues involved in use of porcine dermal collagen. Further, TransCyte® is not suitable for prolonged application because it may result in immunological rejection by the patient. It has also not been established for application in burns of the head or hands, or in surgically excised full-thickness and deep partial-thickness wounds prior to autografting. The nylon mesh used in TransCyte® is also not biodegradable.^{[12]}

Therefore, there is a need to provide a wound dressing material that is cost effective, provides better protection against infections, is suitable for deep wound application and is bioabsorbable. There is also a need to overcome or at least ameliorate one or more of the disadvantages described above.

### SUMMARY

According to a first aspect, there is provided a composite comprising:

a semi-permeable barrier layer that is permeable to oxygen and impermeable to microorganisms; and

a scaffold fiber layer formed by electrospinning fibers on one side of said semi-permeable barrier layer.

In one embodiment, one or more cells, preferably skin cells, are provided in said scaffold fiber layer.

In one embodiment, said scaffold fiber layer comprises gelatin.

According to a second aspect, there is provided a method of making a composite comprising electrospinning fibers on a semi-permeable barrier layer that is permeable to oxygen and impermeable to microorganisms.

In one embodiment, the method comprises the step of seeding one or more cells, preferably skin cells, in said scaffold layer.

In one embodiment, the method comprises the step of providing gelatin in said scaffold layer.

According to a third aspect, there is provided a composite comprising:

a semi-permeable barrier layer that is permeable to oxygen and impermeable to microorganisms;

at least two scaffold fiber layers, said scaffold fiber layers formed by electrospinning fibers on one side of said semi-permeable barrier layer; and

at least one cell provided in each of said at least two scaffold fiber layers, wherein said scaffold fiber layers comprise different cell types.

According to a fourth aspect, there is provided a method of making a composite comprising:
electrospinning fibers on a semi-permeable barrier layer to form a scaffold fiber layer; and
seeding at least one cell in said scaffold fiber layer.

According to a fifth aspect, there is provided use of a composite according to the first aspect or third aspect, for treating a dermal condition of an animal.

According to a sixth aspect, there is provided a method for treating a dermal condition of an animal comprising applying the composite defined in the first aspect, or third aspect, to the skin of the animal.

According to a seventh aspect, there is provided a kit for treating a dermal condition of an animal comprising a composite as defined in the first aspect together with instructions for applying the composite to the skin of an animal having said dermal condition.

### DEFINITIONS

The following words and terms used herein shall have the meaning indicated:

The term “nanofiber” is used to represent one filament or a bundle of filaments having average diameter(s) less than about 1,000 nanometers (nm), for example, below 500 nm, preferably from about 1 nm to 100 nm.

The term “co-axial nanofiber” refers to a nanofiber composed of more than one material wherein the core and shell of the nanofiber are made up of different materials.

The term “conducting fluid” refers to a fluid that is capable of carrying current. It may be a pure liquid, gel or solution. It may be a solution of fiber forming material in a suitable solvent.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### DETAILED DISCLOSURE OF EMBODIMENTS

Exemplary, non-limiting embodiments of a composite, method of preparing the composite and use of the composite will now be disclosed. The embodiments disclosed herein represent an improvement over the prior art wound dressing materials as well as the prior art tissue engineering scaffolds with regards to improved dermal rehabilitation, higher resistance to infections, enhanced cell growth and cost effectiveness.

There is provided a composite comprising a semi-permeable barrier layer that is permeable to oxygen and impermeable to microorganisms; and a scaffold fiber layer formed by electrospinning fibers on one side of the barrier layer.

### Semi-permeable Barrier Layer

The composite comprises a semi-permeable barrier layer that is permeable to oxygen but impermeable to micro-organisms and dust. The semi-permeable barrier layer may be composed of biological, synthetic or a blended materials.

The semi-permeable layer may be composed of a biological material selected from the group consisting of cellulose acetate, phospholipids, cotton and mixtures thereof.

In one embodiment the semi-permeable barrier layer comprises a polymer. The semi-permeable barrier layer may be composed of a material selected from the group consisting of polycellulose, polyurethane, polystyrene, polyimides, polyamides, resins, nylon, silicon, polyester, polyolefin (such as polyethylene, polypropylene, polybutylene), polyamide, polysilicone, copolymers and mixtures thereof.

The semi-permeable barrier layer may be composed of blended materials that are composites or mixtures of biological and synthetic materials.

The semi-permeable barrier layer may be a commercially available wound dressing membrane such as Tegaderm™ from 3M Health Care Ltd, Dermagraft™ from Smith & Nephew, Transcyte™ from Smith & Nephew and Integra™ patches from Integra LifeSciences Holdings Corporation and Biobrane™ adhesive patches from Bertek Pharmaceuticals Inc.

In one embodiment, the semi-permeable barrier layer is Tegaderm™.

The semi-permeable material may be removably attached to the scaffold fiber layer using an adhesive. The semi-permeable barrier layer may be partially or completely coated with an adhesive material. The adhesive material coating may be present on one side or both sides of the semi-permeable barrier layer. The adhesive material may be any material that does not allow growth of micro-organisms and is non-toxic to subject animals or human beings. The adhesive material may be composed of biological, synthetic or blended materials.

The adhesive material may be selected from the group consisting of gelatin material, resin based adhesives, phenol based adhesives, aldehyde based adhesives and mixtures thereof.

Preferably, said semi-permeable barrier layer is Tegaderm™, having substantially no acrylic adhesive. The Tegaderm™ may have had the acrylic adhesive therefrom.

The thickness and average pore size of the semi-permeable barrier layer may be suitably chosen to allow permeation of oxygen and to restrict permeation of micro-organisms.

The average pore size of the semi-permeable barrier layer may be in a range selected from the group consisting of 1 μm to 50 μm, 2 μm to 40 μm, 3 μm to 30 μm, 4 μm to 20 μm and 5 μm to 10 μm.

In one embodiment, average pore size of the semi-permeable barrier layer is in the range of 5 μm to 10 μm. The thickness of the semi-permeable barrier layer may be in the range selected from the group consisting of about 0.005 mm to about 2 mm, about 0.005 mm to about 1 mm, about 0.005 mm to about 0.5 mm, about 0.005 mm to about 0.1 mm, about 0.005 mm to about 0.05 mm, about 0.005 mm to about 0.02 mm, about 0.01 mm to about 0.02 mm, and about 0.015 mm to about 0.02 mm. In one embodiment, the thickness of the semi-permeable barrier layer is 0.018 mm.

### Scaffold Fiber Layer

The composite comprises a scaffold fiber layer formed by electrospinning fibers on one side of the semi-permeable barrier layer. The scaffold fiber layer may be produced using the apparatus of FIG. 1.

The conducing fluid used in the electrospinning process may be solution of fiber forming material in a suitable solvent. The solvent may be selected from the group consisting of alcohols, ketones, aldeydes and alkyl halides. Exemplary solvents include methanol, ethanol, acetone, chloroform, glycerol, dimethylformamide, dichloromethane, tetrahydrofuran, methylene chloride, 2,2,2-trifluoroethanol and mixtures thereof. Respectively suitable solvents will change depending on type of solute used. A simple review can be obtained from Z. M. Huang et al. Composite Science and Technology 63 (2003 2223-2253], which is incorporated in its entirety herein. In one embodiment the solvent is a mixture of chloroform and methanol in a ratio of 3 parts of chloroform to 1 part of methanol.

The scaffold fiber layer may be composed of any material that is not toxic to the subject animal. The scaffold fiber layer may be composed of any biological, synthetic or blended material that can be spun into fibers. The scaffold fiber layer may be composed of a biodegradable and bioabsorbable material.

The scaffold fiber layer may be composed of a material selected from the group consisting of collagen, gelatin, keratin, chitosan, polypeptides, proteins, poly-ε-caprolactone (PCL), polyethylene oxide, polyvinyl alcohol, polyvinyl pyrolidon, polyamide, polylactic acid and mixtures thereof.

In one embodiment the scaffold fiber layer is composed of poly-ε-caprolactone. Other suitable materials for the scaffold fiber layer are disclosed in Z. M. Huang et al. Composite Science and Technology 63 (2003 2223-2253.

In one embodiment the scaffold fiber layer is composed of gelatin. The gelatin may be selected from the group consisting of bovine collagen, porcine collagen, ovine collagen, equine collagen, synthetic collagen, agar, synthetic gelatin, and combinations thereof.

In another embodiment, the scaffold fiber layer is composed of fibers formed by electro-spinning a mixture of materials. In one embodiment, one of the fibers forming materials may be a medicinal agent.

In yet another embodiment, the scaffold fiber layers is composed of fibers comprising two materials, the first material forming a shell and the second material forming the core of the fiber. Such fibers may be called as co-axial fibers. A scaffold fiber layer comprising co-axial nanofibers may be produced by using apparatus of FIG. 1 together with the syringe needle of FIG. 2.

In one embodiment, at least one of the fibers forming materials used for producing co-axial nanofibers may be a medicinal agent.

The diameters of the core and the shell in a co-axial nanofiber may depend upon the concentration of the fibers forming material in the conducting fluid.

The fibers of the scaffold layer may be macro, micro, nano or mixed fibers. The scaffold layer may comprise multiple sub-layers wherein each sub-layer is selected from the group consisting of macro, micro and nano fibers.

In one embodiment the scaffold fiber layer is a nano-fiber layer.

The thickness of the scaffold fiber layer may be suitably chosen depending upon the application. The thickness of the scaffold layer may be selected from the group consisting of about 0.05 mm to about 5 mm, about 0.05 mm to about 4 mm, about 0.05 mm to about 3 mm, about 0.05 mm to about 2 mm, about 0.05 mm to about 1.5 mm, about 0.08 mm to about 1.5 mm, about 0.1 mm to about 1.5 mm, about 0.2 mm to about 1.5 mm, about 0.5 mm to about 1.5 mm, about 0.8 mm to about 1.5 mm. In one embodiment, the thickness of the scaffold fiber layer is about 1 mm.

The average pore size of the scaffold fiber layer and the pore size distribution may be suitably chosen depending upon the application. For example, when the scaffold is used for tissue engineering applications, the average pore size is chosen based on the size of the cells to be cultured onto the scaffold.

The scaffold fiber layer may be capable of supporting cell attachment and proliferation therein. The scaffold fiber layer may be seeded with cells selected the group consisting of embryonic stem cells, embryonic germ stem cells, fetal tissue derived epithelial cells, mesenchymal cells, endothelial stem/progenitor cells, bone marrow derived mesenchymal stem/progenitor cell, umbilical cord blood derived mesenchymal stem/progenitor cells, adipose tissue derived mesenchymal stem/progenitor cells, hair follicular epidermal stem cells, limbal epithelial stem cells, limbal epithelial stem cells, nail bed germ cells, osteoblast cells, chondrocytes, smooth muscle cells, tenocytes, buccal and oral mucosa keratinocytes and fibroblast cells, ligament fibroblast cells and periodental ligament fibroblasts cells.

In one embodiment, the cells are skin cells selected from the group consisting of keratinocytes, dermal fibroblasts, melanocytes and combinations thereof.

In one embodiment, the scaffold layer is seeded with Human Dermal Fibroblast cells.

### Electrospinning

The scaffold fiber layer of the composite is prepared by electrospinning fibers on one side of the semi-permeable barrier layer.

The structure of the scaffold fiber layer may depend upon the electrospining parameters such as electric field strength, length of the electric field, length and radius of the syringe needles and fiber forming solution flow rate.

The strength of electric field applied to the scaffold forming solution may be in the range selected from the group consisting of 5 kV to 25 kV, 5 kV to 20 kV, 5 kV to 15 kV, 5 kV to 10 kV, 6 kV to 15 kV, 6 kV to 14 kV and 8 kv to 12 kv.

The length of electric field applied to the scaffold forming solution may be in the range selected from the group consisting of about 5 cm to about 25 cm, about 5 cm to about 20 cm, about 5 cm to about 15 cm, about 5 cm to about 10 cm, about 5 cm to about 25 cm, about 10 cm to about 25 cm and about 10 cm to about 15 cm.

The radius of syringe needle used for electrospinning the scaffold fiber barrier may be in the range selected from the group consisting of about 0.1 mm to about 2 mm, about 0.1 mm to about 1 mm, about 0.1 mm to about 0.5 mm, about 0.1 mm to about 0.3 mm, about 0.2 mm to about 2 mm, about 0.2 mm to about 1.2 mm. In one embodiment, the radius of syringe needle used for electrospinning the scaffold fiber barrier is about 0.21 mm.

The structure of the scaffold fiber layer may also depend upon parameters of the fiber forming solution such as concentration of the solution, density of the solution, viscosity of the solution, ionic strength of the solution, resistivity of the solution and conductivity of the solution.

The properties of the fiber forming solution, such as concentration, viscosity etc will be dependent on the solvent used. Properties of electrospinning solutions are disclosed in S. H Tan et al. Polymer 46 (2005) 6128-6134, which is incorporated herein in its entirety.

### Uses of the Composite

There is provided use of the composite for treating a dermal condition of an animal. The composite used for treating a dermal condition may be a cell seeded composite. In one embodiment a composite seeded with HDF cells is used to treat the dermal conditions.

The dermal condition may be a burn on an animal's skin. The composite may used to treat a burn that extends to at least the epidermis of the animal's skin. The composite may also be used to treat a burn that extends to the dermis or the subcutaneous fat region of an animal's skin.

There is provided a method for treating a dermal condition of an animal comprising applying multiple layers of a cell seeded composite on to the wound wherein the layers are applied one at a time with suitable time interval between application of two successive layers. The number of cell seeded composite layers applied to the affected area may depend up on the depth of the wound.

The time interval between application of two successive composite layers may be selected from the group consisting of 1 day to 30 days, 7 days to 21 days, 7 days to 14 days, 14 days to 17 days.

In one embodiment, a first layer composite seeded with HDF cells is applied on to the wound and allowed to remain in that position for 15 days. After 15 days the semi-permeable barrier layer of the composite is peeled off and another layer of HDF seeded composite is applied to the wound area, on the top of the scaffold layer of the first composite layer. The process is repeated until a 75% dermal reconstitution is achieved. Autologous dermal graft is them applied to the wound area. The method is may be called as Autologous Layered Dermal Reconstitution (ALDR).

There is also provided a use of the composite for drug delivery applications. A medicinal compound may be embedded in the scaffold fiber layer. The medicinal compound may be soluble in the body fluids or may be magnetically or electrically detachable from the scaffold layer. The composite may be applied to the area suitable for drug delivery. The adhesive coating on the semi-permeable barrier layer may provide physical stability to the composite.

The composite may be used for cell delivery applications. The cells of interest may be cultured on the scaffold fiber layer of the composite. The composite containing cells may be applied to the area where the cells need to be delivered.

### BRIEF DESCRIPTION OF DRAWINGS

Embodiments will now be described with reference to the following drawings.

FIG. 1 is a schematic diagram of electrospinning apparatus used to produce a nanofiber scaffold layer in accordance with a disclosed embodiment.

FIG. 2 is a schematic diagram of syringe needle used for producing co-axial nanofibers in accordance with a disclosed embodiment.

FIG. 3 is a schematic diagram of cross section of a co-axial nanofiber in accordance with a disclosed embodiment.

FIG. 4a is a SEM (Scanning Electron Microscope) image of nanofiber scaffold layer of a composite prepared in accordance with a disclosed embodiment. The image was taken at a resolution of 3600×.

FIG. 4b is a SEM image of nanofiber scaffold layer of a composite prepared in accordance with a disclosed embodiment.

The image was taken at a resolution of 14400×.

FIG. 5 is a SEM image of human epidermal keratinocyte cells cultured on a Tegaderm™ wound dressing material. The image was taken at a resolution of 1311×.

FIG. 6 is a SEM image of human dermal fibroblast cells cultured on a Tegaderm™ wound dressing material. The image was taken at a resolution of 1106×.

FIG. 7 is a bar graph illustrating the difference between growth of human epidermal keratinocytes cultured on Tegaderm™ wound dressing material and on tissue culture plastics. The growth was assessed by means of an MTS assay.

FIG. 8 is a bar graph illustrating the difference between growth of human dermal fibroblasts cultured on Tegaderm™ wound dressing material and on tissue culture plastics. The growth was assessed by means of a MTS assay.

FIG. 9 is a bar graph illustrating the difference between growth of Human Dermal Fibroblast (HDF) cells cultured on a Tegaderm™-nanofiber (TG-NF) composite in accordance with a disclosed embodiment and on a poly-ε-caprolactone (PCL) scaffold. The growth was assessed by means of a MTS assay.

FIG. 10 is a bar graph illustrating the difference between growth of HDF cells cultured on a TG-NF composite in accordance with a disclosed embodiment and a PCL scaffold. The growth was assessed by cell counting.

FIG. 11a is a FESEM (Field Emission Scanning Electron Microscope) image of HDF cells on day 3 of culture on a PCL scaffold.

FIG. 11b is a FESEM image of HDF cells on day 3 of culture on a TG-NF composite in accordance with a disclosed embodiment.

FIG. 11c is a FESEM image of HDF cells on day 7 of culture on a PCL scaffold.

FIG. 11d is a FESEM image of HDF cells on day 7 of culture on a TG-NF composite in accordance with a disclosed embodiment.

FIG. 11e is a FESEM image of HDF cells on day 21 of culture on a PCL scaffold.

FIG. 11f is a FESEM image of HDF cells on day 21 of culture on a TG-NF composite in accordance with a disclosed embodiment.

FIG. 12a is a light microscope image of HDF cells on Day 1 of culture on a PCL scaffold.

FIG. 12b is a light microscope image of HDF cells on Day 1 of culture on a TG-NF composite in accordance with a disclosed embodiment.

FIG. 12c is a light microscope image of HDF cells on Day 7 of culture on a PCL scaffold.

FIG. 12d is a light microscope image of HDF cells on Day 7 of culture on a TG-NF composite according to a disclosed embodiment.

FIG. 12e is a light microscope image of HDF cells on Day 21 of culture on a PCL scaffold.

FIG. 12f is a light microscope image of HDF cells on Day 21 of culture on a TG-NF composite according to a disclosed embodiment.

FIG. 13 illustrates the steps of autologous layered dermal reconstitution (ALDR) method according to a disclosed embodiment.

FIG. 14 are FESEM morphological images of a gelatin/PCL composite nanofiber scaffold of magnification (a) 3,000×, (b) 8,000×, (c) 12,000× and (d) 20,000×.

FIG. 15(A) shows a FESEM cross sectional views of a PCL-Gelatin nanofiber scaffold through freeze fracturing at a magnification of 750×.

FIG. 15(B) shows a FESEM cross sectional views of a PCL-Gelatin nanofiber scaffold through freeze fracturing at a magnification of 1500×.

FIG. 16 shows a stress-strain curve of a gelatin/PCL nanofibrous scaffold under tensile loading before and after detachment from Tegaderm™ wound dressing

FIG. 17 shows a stress-strain graph of gelatin/PCL nanofibrous scaffold intact with Tegaderm™ wound dressing.

FIG. 18 shows a graph of the viability of HDFs on TCPS, PCL NFM and PCL-Gelatin NFM. Cells were seeded at density of 1.5×10⁴ cells/well and cultured for a period of 7 days.

FIG. 19 is a graph showing attachment of HDFs on TCPS, PCL NFM and PCL-Gelatin NFM. Cells were seeded at density of 3×10⁴ cells/well.

FIG. 20 shows the cell count of HDFs on TCPS, PCL NFM and PCL-Gelatin NFM. Cells were seeded at density of 1.5×10⁴ cells/well and cultured for a period of 7 days.

FIG. 20 show FESEM morphological views of HDF proliferation on gelatin/PCL composite scaffold: (a) Day 1, (b) Day 3, (c) Day 5, (d) Initial HDF penetration into scaffold structure.

### DETAILED DESCRIPTION

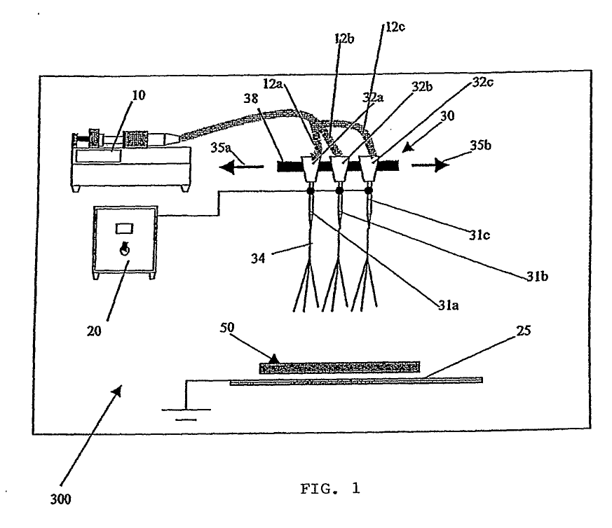

FIG. 1 is a schematic diagram of an electrospinning apparatus used to produce a nanofiber scaffold layer. The electrospinning system 300 comprises a syringe pump 10, a high voltage power supply 20, a movable multiple spinneret system 30 and a nanofiber collector 25. The syringe pump 10 feeds a conducting fluid used for forming nanofibers to the multiple spinneret system 30 through a series of tubes (12a, 12b, 12c).

A plurality of spinnerets comprising three syringe needles (31a, 31b, 31c) is mounted on the multiple spinneret system 30. Each of the three syringe needles (31a, 31b, 31c) is mounted on to a spinneret holder 38 by means of respective plugs (32a, 32b, 32c).

In use, the conducting fluid flows from the pump 10 through the series of tubes (12a, 12b, 12c), into each of the three syringe needles (31a, 31b, 31c) via the plugs (32a, 32b, 32c). The multiple spinneret system 30 is operable to move in a reciprocating manner, for example, from left to right, as indicated by arrows (35a and 35b).

A grounded collector 25 is positioned below the syringe needles (31a, 31b, 31c) to create an electric field between the charged syringe needless (31a, 31b, 31c) and the collector 25. The electric field causes tiny jets 34 of the conducting fluid to be ejected from the tip of each syringe needle (31a, 31b, 31c). The jets 34 are deposited onto the collector 25, and form a scaffold of nanofibers on the collector 25. A semi-permeable barrier layer 50 may be located on the collector 25 to form the scaffold fiber layer onto the semi-permeable barrier layer 50.

The structure and working of apparatus of FIG. 1 is described in further detail in Singapore Patent Application No. SG 200403355-1.

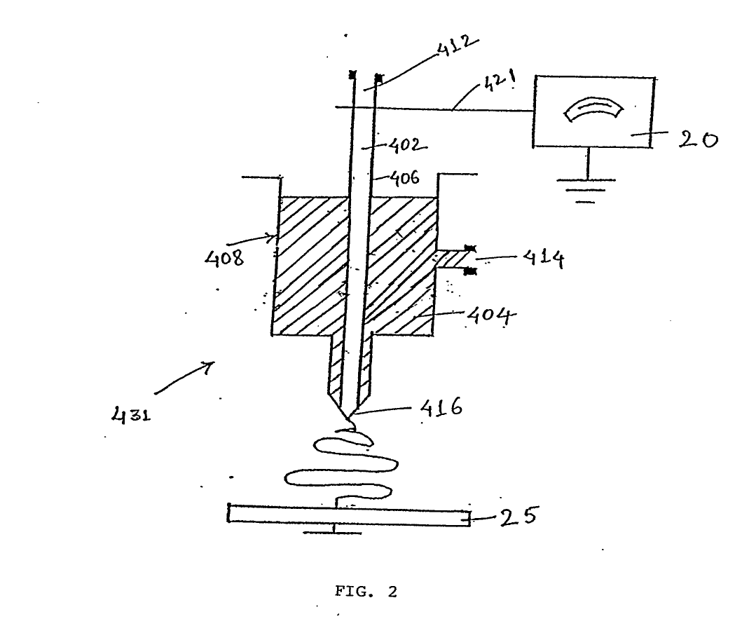

FIG. 2 represents another embodiment of the syringe needle 31. FIG. 2 shows schematic diagram of a syringe needle 431 that can be used in place of the syringe needle 31 in the electrospinning apparatus of FIG. 1.

The syringe needle 431 comprises two capillary tubes 406 and 408 arranged concentrically. The capillary tubes (406,408) are made up of stainless steel. The syringe needle 431 is provided with two inlets 412 and 414 for supplying a first conducting fluid and a second conducting fluid to the syringe needle 431. The concentric arrangement of the capillary tubes (406,408) forms two separate channels 402 and 404 for the passage of the first conducting fluid and the second conducting fluid from the respective inlets (412,414) to the tip of the syringe 431.

The inlets (412,414) are connected to two different syringe pumps (not shown) by means of teflon tubes (not shown). The syringe pumps used in this embodiment are identical to the syringe pump 10 of FIG. 1 and the tubes used in this embodiment are identical to tube 12 of FIG. 1. The syringe 431 is connected to the power supply 20 by means of a copper wire 420.

In use, two different conducting fluids are pumped into the syringe needle 431 by the respective pumps. When electric field is applied to the syringe needle 431, jets of conducting fluids are ejected from the needle and are deposited on to the grounded collector in the form of nanofibers.

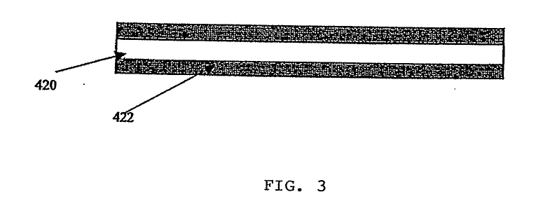

FIG. 3 shows a schematic diagram of a nanofiber 430 produced by using the syringe needle 431 of FIG. 2. The nanofibers produced by using syringe needle 431 are composed of two fiber-forming materials. The first material corresponds to the first conducting fluid and the second material corresponds to the second conducting fluid. The first material forms core 420 of the nanofiber 430 and the second material form the shell 422 of the nanofiber 430.

### Use of Composite

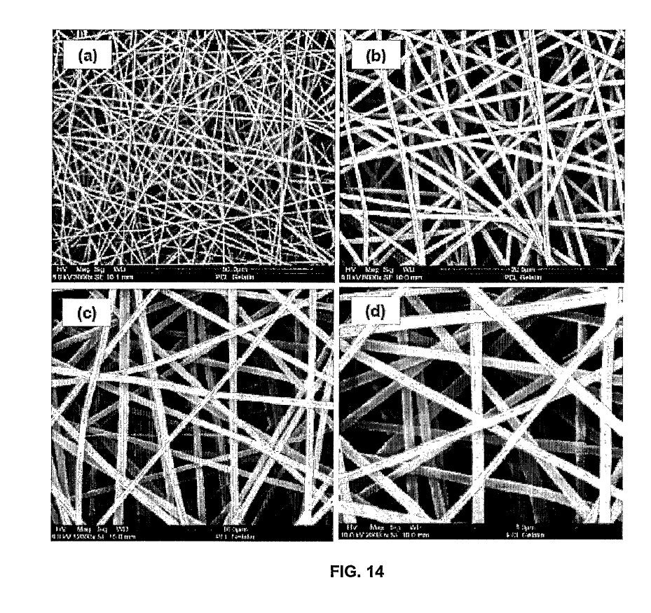

A composite manufactured as described above is able to be used for treating a dermal condition. Referring to FIG. 13 there is shown steps of an autologous layered dermal reconstitution (ALDR) method.

In Step 1 a first TG-NF composite 10 comprising scaffold 14 of poly-ε-caprolactone (PCL) that has been electrospun onto a layer of Tegaderm™ would dressing 12 is placed onto a patient's burnt skin wound 8. The scaffold layer 14 of the TG-NF composite 10 is seeded with Human Dermal Fibroblast (HDF) cells.

In Step 2 the TG-NF composite 10 seeded with HDF cells, is left on the skin 8 for a period of time to allow skin healing assisted by the HDF cells as they proliferate within the scaffold 14 in situ.

In Step 3 the layer of Tegaderm™ 12 is peeled off the scaffold layer 14.

In Step 4, directly after step 3, a second TG-NF composite 10A comprising scaffold 14A of poly-ε-caprolactone (PCL) that has been electrospun onto a layer of Tegaderm™ would dressing 12A is placed onto the first scaffold layer 14 so that the second scaffold 14A is in direct contact with the in-situ scaffold 14.

Additional layers may be placed on the skin by repeating steps 3 and 4 until 70-80% of the original dermal thickness is reconstituted on the patient's skin 8.

Finally, in optional step 8, a skin graft 16 is placed on top of the last remaining scaffold 14A.

### EXAMPLES

Non-limiting examples of the invention, and a comparative example will be now be described in greater detail by reference to specific Examples, which should not be construed as in any way limiting the scope of the invention.

### Example 1

### Keatinocyte-Tegaderm™

It has long been assumed that the adhesive in polyurethane materials, such as Tegaderm™, would lead to cell death. However, the inventors have surprisingly found that Keatinocyte cells can not only survive but can in fact grow directly on Tegaderm™.

Human epidermal keatinocyte cells were seeded directly onto a layer of Tegaderm™ and cultured in an assay as described in T. T. Phan [33].

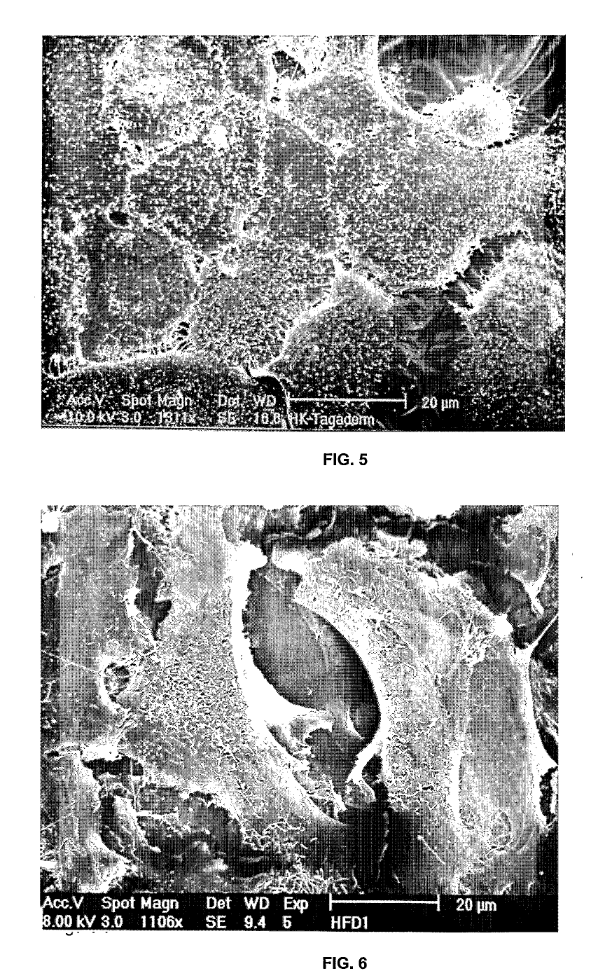

FIG. 5 shows a SEM image of the human epidermal keratinocyte cells cultured direct on Tegaderm™ wound dressing material. The image was taken at a resolution of 1311×.

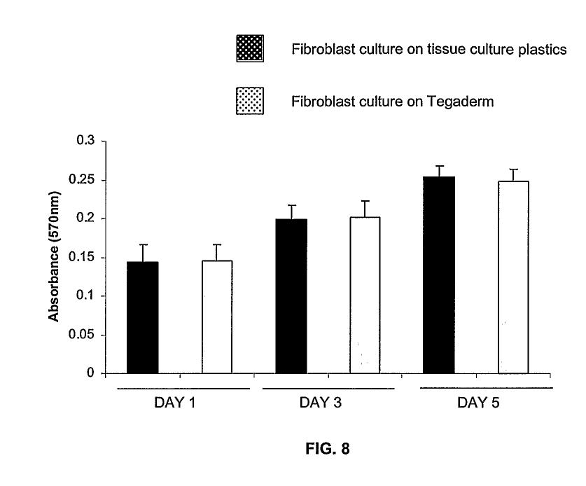

FIG. 7 is a bar graph illustrating the difference between growth of human epidermal keratinocytes cultured on Tegaderm™ wound dressing material and on tissue culture plastics as disclosed in T. T. Phan et al. The growth was assessed by means of an MTS assay. FIG. 7 shows that cell proliferation on Tegaderm™ wound dressing material is comparable to cell culturing in tissue culture. Accordingly, the inventors have found that Tegaderm™ membrane material can be used to support cell proliferation and is therefore a suitable material for use in a composite for treating dermal conditions.

### Example 2

### Human Dermal Fibroblast Cells-Tegaderm™

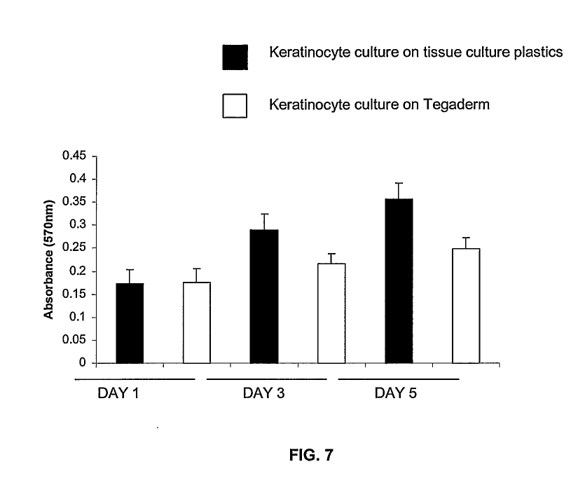

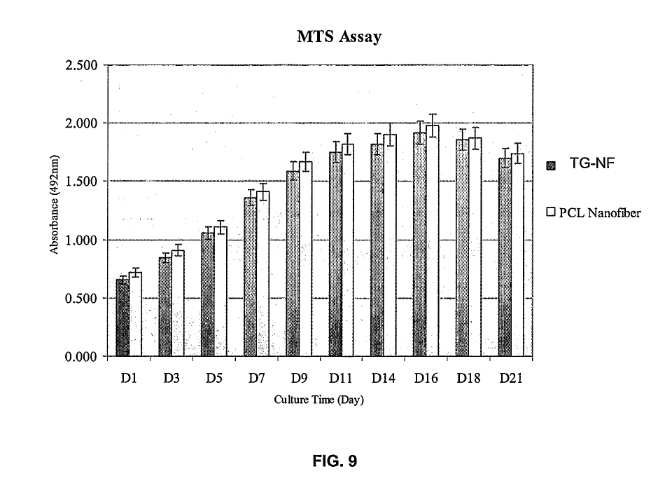

Example 1 was repeated only in this example, Human Dermal Fibroblast (HDF) Cells were seeded directly onto a layer of Tegaderm™ rather than keratinocyte cells. FIG. 6 shows a SEM image of human dermal fibroblast cells cultured on the Tegaderm™ wound dressing material. The image was taken at a resolution of 1106×. FIG. 8 is a bar graph illustrating the difference between growth of the HDF cells cultured on Tegaderm™ wound dressing material and on tissue culture plastics. The growth was assessed by means of a MTS assay. Again, the inventors have found that Tegaderm™ membrane material can be used to support HDF cell proliferation and is therefore a suitable material for use in a composite for treating dermal conditions.

### Example 3

### Manufacture of a Tegaderm™-PCL Composite

A composite was prepared according a disclosed embodiment. In this composite, Tegaderm™ wound dressing material was employed as a semi-permeable barrier layer. Using the apparatus of FIG. 1, a nanofiber scaffold layer was electrospun onto the Tegaderm™ material. An electric field strength of 10 kV was used, a needle radius of 0.21 mm, a spinning solution feed rate of 0.8 ml/hr and an electric field distance of 12 cm. Poly-ε-caprolactone (PCL) was used as the fiber forming material. The conducting solution was prepared by dissolving poly-ε-caprolactone (PCL) in a mixed solvent of chloroform and methanol (3 volume of chloroform: 1 volume of methanol) to form a 10 wt % PCL solution. The conducting solution was used to form nanofibers onto the Tegarderm™ material. The composite thus prepared is called as TG-NF composite.

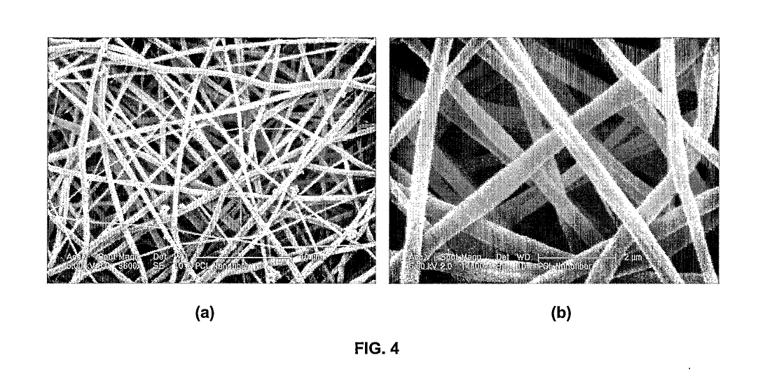

Using a SEM (Scanning Electron Microscope), images of the scaffold fiber layer of the composite were taken at various resolutions. FIG. 4a is a SEM (Scanning Electron Microscope) image of the scaffold fiber layer of the formed composite. The image was taken at a resolution of 3600×. FIG. 4b is another SEM image of the scaffold fiber layer of the composite. The image was taken at a resolution of 14400×.

### Example 4

### Seeding of Human Dermal Fibroblast Cells onto Composite

Human Dermal Fibroblast (HDF) cells were seeded onto the scaffold fiber layer of the TG-NF composite of Example 3. The cells were allowed to proliferate for a period of 21 days. The HDF cells were obtained from an 8-month-old Chinese infant (Cell Research Corporation). The HDFs were plated as a monolayer and cultured to confluence in DMEM containing 10% FBS (Fetal Bovine Serum) and 1% antibiotic solution (penicillin-streptomycin). Media was replaced every 3 days and the cultures were maintained in a humidified incubator at 37° C. with 5% CO₂. All culture media and reagents were purchased from Research Biolabs (Sigma, St Louis, Mo., USA).

The growth of cells on the scaffold layer was assessed using standard MTS (3-(4,5-dimethylthiazol-2-yl)-5-(3-carbomet and also by hoxyphenyl)-2-(4-sulfophenyl-tetrazolium innersalt) assay standard cell count method. To study cell proliferation on the substrates, viable cells was determined by using the calorimetric MTS assay (CellTiter 96® A_{Queous}Assay, Madison, Wis., USA). The mechanism behind this assay is that metabolically active cells will react with the tetrazolium salt in the MTS reagent to produce a soluble formazan dye that can be absorbed at 492 nm. The substrates were rinsed with PBS, followed by incubation with 20% MTS reagent in serum-free culture medium for 3 hours. Thereafter, aliquots were pipetted into a 96-well plate. The 96-well plate was then placed into a spectrophotometric plate reader (FLUOstar OPTIMA, BMG Lab technologies, Germany) and the absorbance at 492 nm of the content of each well was measured. In order to count the number of viable cells attached to the PCL scaffolds and TG-NF constructs, the substrates were harvested, washed with PBS to remove non-adherent cells, and then incubated in 0.5 ml of 1× trypsin at 37° C. for 5 min. The trypsinization process was stopped by adding 0.5 ml of DMEM to each sample. The cell numbers were then counted using a hematocytometer and microscope.

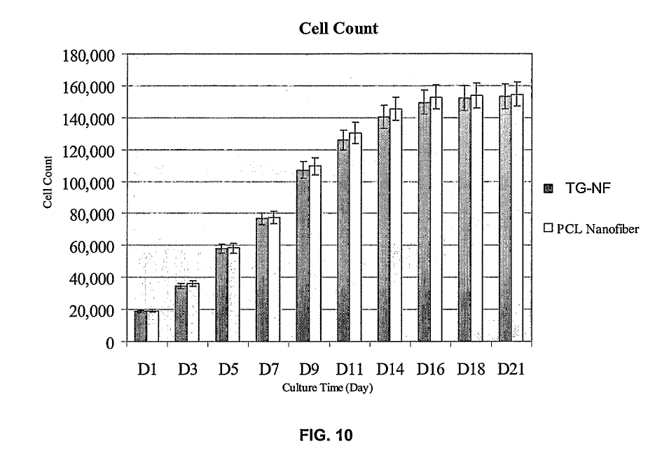

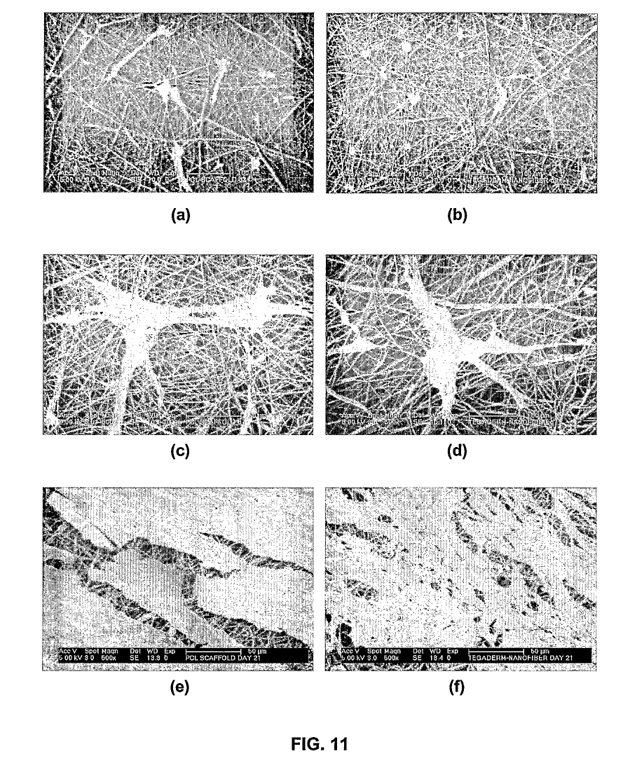

FIG. 9 shows a comparison between growth of HDF cells cultured on a Tegaderm™-nanofiber (“TG-NF”) composite of Example 1 and growth of HDF cells cultured on PCL scaffold without Tegaderm™ (“PCL Nanofiber”). The growth was assessed by means of a MTS assay as outlined in Example 2 above. HDF proliferation on PCL Nanofiber scaffolds and TG-NF constructs was studied at Days 1, 3, 5, 7, 9, 11, 14, 16, 18 and 21, with the results shown in FIGS. 9 and 10. Both the optical density and number of cells were noted to have increased significantly through the 21-day span, demonstrating that cell proliferation occurred successfully on both types of substrates and that cell proliferation was not adversely affected by the presence of the Tegaderm™ would dressing material.

In FIG. 9, it was observed that optical density of HDFs within PCL Nanofiber and TG-NF kept increasing until Day 16 when it started decreasing. This was because HDFs had proliferated till confluence at Day 16 when there were no more available spaces on the constructs for further proliferation.

The results were substantiated by the observations made in FIG. 10, which shows a comparison between growth of HDF cells cultured on a TG-NF composite of Example 1 and growth of cells cultured on the PCL Nanofiber scaffold. The growth was assessed by cell counting.

FIG. 10 shows that the cell counts were noted to have reached a plateau from Day 16. The cell counting results are in agreement with the assessment by MTS assay. At every time point, the numbers of cells present on the PCL nanofiber scaffold and TG-NF construct were comparable, demonstrating that cell growth was comparable on both the substrates. Therefore, it can be concluded that the TG-NF construct is a suitable non-toxic substrate for cell growth and proliferation.

### Protocol of Scanning Electron Microscopy Imaging:

For characterization, the PCL nanofiber and TG-NF constructs were sputter coated with gold (BAL-TEC; SCD 005 Sputter Coater; Germany). Morphological imaging of the constructs was performed using Field Emission Scanning Electron Microscopy (FEI Co.; XL30 FEG SEM; USA) at an accelerating voltage of 15 kV.

### Analysis:

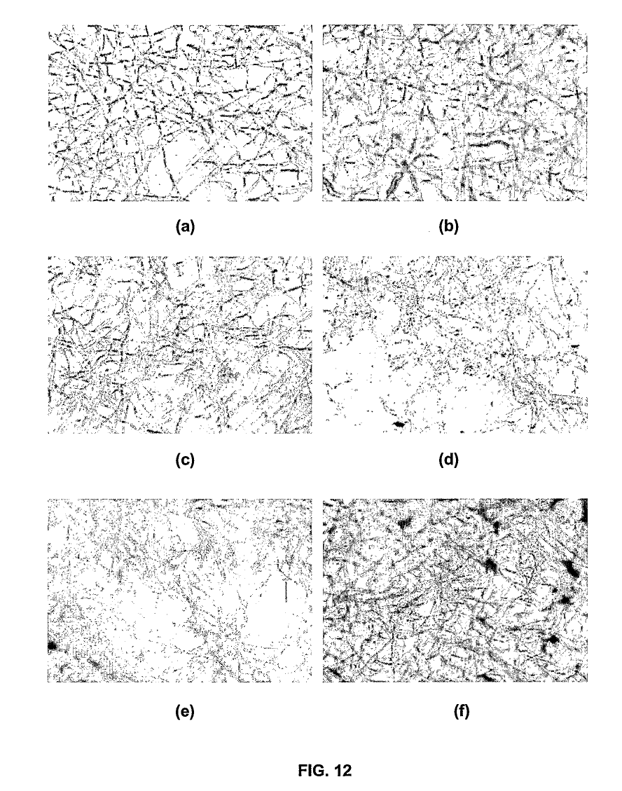

Cell morphology on the PCL scaffolds and TG-constructs was studied by FESEM at Days 3, 7, and 21. It was noted that at Day 3 (refer to FIG. 11a and 11b) HDFs only reached approximately 10% confluence. This was due to the low HDF seeding density used in this study in order to observe proliferation over the 21 day span. The cells were seen to be characteristically spindle shaped and stretched across the nanofibrous substrates during the course of proliferation.

Subsequently at Day 7 (FIG. 11c and 11d), it was observed that the HDFs had increased in number and reached about 30% confluence on both substrates. HDF proliferation and growth continued progressively and at Day 21 (FIG. 11e and 11f), HDF proliferation had almost reached 100% confluence. Both the PCL scaffold and TG-NF construct were almost completely covered with a continuous HDF monolayer. Cracks were observed from the micrographs at this point. This could have been due to the contractile force exerted by the HDF monolayer during the sample preparation phase for FESEM, where the fibroblasts populated substrates were dehydrated with increasing grades of ethanol solutions and air-dried overnight.

From the FESEM micrographs of FIG. 11, it was established that there was successful HDF proliferation and adherence on both nanofibrous substrates. Comparing the FESEM micrographs taken for both the PCL scaffolds and TG-NF constructs at each time point, it was observed that the density of HDFs on both substrates was always comparable and that the cells reached almost 100% confluence on each at an near equivalent rate.

From this experimental data, it can be seen that the TG-NF construct can be considered a suitable host substrate for fibroblast population, with results of cell proliferation was much comparable to that of the PCL nanofibrous scaffold.

### Protocol for Histology Sectioning and Imaging:

The PCL nanofiber scaffolds and TG-NF constructs were fixed in 4% formalin and stained with hematoxyline and eosin (H&E). Thereafter, the individually stained substrates were embedded between two layers of OCT embedding medium (Leica; Germany) by immersing the whole structure in liquid nitrogen. Serial sections (5 μm) were sliced using a cryostat (Leica CM3050S; Germany) and examined under an inverted optical microscope (Leica DM IRB; Germany).

### Analysis:

Since fibroblasts are anchorage-dependent cells, it was speculated that high specific surface properties and porosity of the electrospun nanofibrous constructs may aid in the attachment and migration of the cells and its extracellularmatrix (ECM) within the substrate.

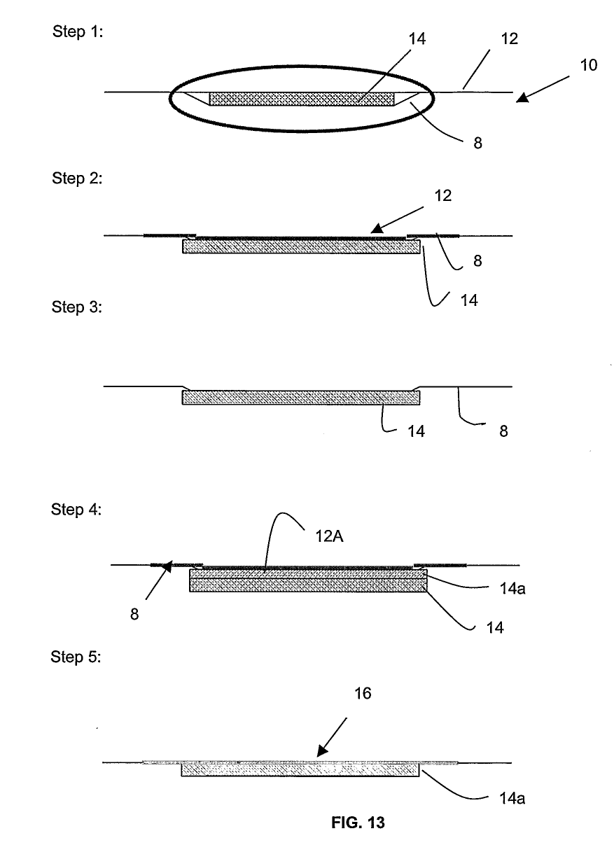

Histological examination revealed ECM integration (light orange stains) inside the PCL scaffolds and TG-NF constructs, which were H&E-stained at Days 1, 7, and 21 (Refer to FIG. 12).

For the individual substrates, it was observed that the density of the HDFs formed had increased over the four time points. This illustrated that not only did the HDFs attach themselves to the surface of the substrates, their ECM have also managed to successfully migrate into its substance as well. This verifies that both the PCL nanofiber scaffold and the TG-NF construct can be further looked into as effective three-dimensional scaffolds for HDF growth and population. However, the scaffolds and constructs produced by the electrospinning techniques were non-woven. Therefore, the porosity and pore sizes were generally irregular and randomly distributed throughout the entire substrate. This provided a better understanding as to why ECM migration and integration was predominantly observed only in certain areas of the substrate and not throughout. Nevertheless, this shortcoming can be rectified with recent developments using aligned nanofibers to fabricate scaffolds, where porosity and pore sizes can be readily controlled.

Another interesting point to note was that at any time point, the densities of HDF in both substrates were relatively comparable. This demonstrates that HDF ECM growth and migration within the TG-NF construct is comparable to the PCL Nanofiber scaffold, which can been regarded as a suitable biodegradable scaffold for a skin substitute.

### Example 5

### Fabrication of Gelatin/PCL Composite Nanofibrous Scaffold

With reference to Y. Z. Zhang et al [26], where Bone Marrow Stromal Cells (BMSC) are noted to be able to proliferate within a gelatin/PCL composite scaffold to a depth of 114 μm. The inventors investigated the use of a gelatin and PCL polymer as a suitable scaffold material for cell proliferation and penetration. Gelatin has numerous merits which include biological origin, biodegradability, biocompatibility and commercial availability at relatively low cost. Gelatin has also established itself to be used widely in the pharmaceutical and biomedical field as wound dressings, carrier for drug delivery and sealants [26-27].

The composite material was fabricated using gelatin Type A (300 bloom, Sigma, St. Louis, Mo.) from porcine skin in powder form, PCL (Mn 80,000, Aldrich) with solvent 2,2,2-trifluoroethanol (TFE) (purity ≧99.0%, Fluka, Buchs, Switzerland).

Solutions of gelatin/TFE and PCL/TFE mixtures were prepared in 10% wt concentration and subsequently mixed together in 50:50 ratio under gentle stirring to obtain the gelatin/TFE/PCL solution to be used for fabricating the composite fibrous scaffold using the apparatus of FIG. 1 under the same operating parameters as Example 3 above to produce the gelatin/TFE and PCL/TFE Tegaderm™ composite (“gelatin/TFE/PCL Tegaderm™ composite”).

### Dimensional Property Characterization

The diameter of the gelatin/TFE/PCL Tegaderm™ composite nanofibers was noted in the range of 300-700 nm (80% of nanofibers) with a mean diameter of 500±120 nm using an image analysis software (ImageJ, National Institute of Health, USA). Through 3 hours of electrospinning, a nanofibrous mat with approximately 30 μm thickness was obtained. With a known bulk density of (1.34 g/cm³), the porosity of the gelatin/TFE/PCL Tegaderm™ composite nanofiber scaffold can be obtained from the equation:

porosity=(1−d/D)×100%

where d and D represents the apparent density and bulk density respectively [28].

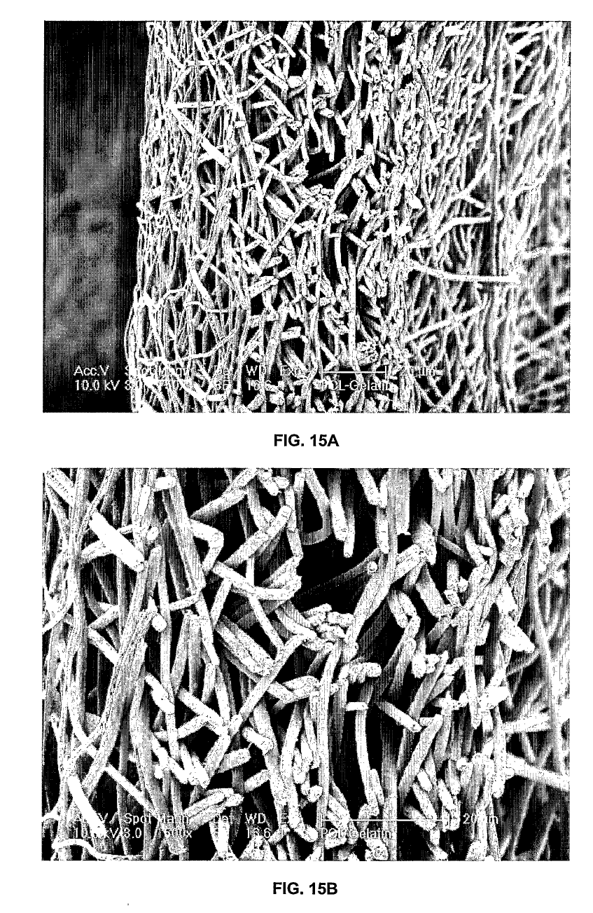

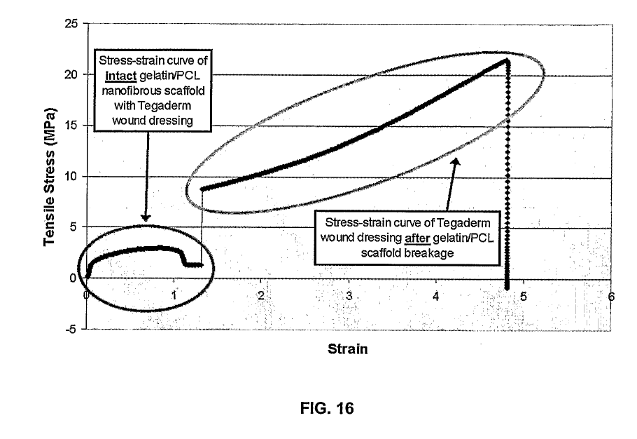

FIG. 14 shows the FESEM morphological images of the gelatin/TFE/PCL Tegaderm™ composite at a magnification of (a) 3,000× (b) 8,000× (c) 12,000× and (b) 20,000×. FIG. 15 shows FESEM cross-sectional views of gelatin/TFE/PCL Tegaderm™ composite through freeze fracturing at a magnification of (a) 750× and (b) 1,500×.

Details results are shown in Table. 1 below.

| | | Mass per unit | Apparent | |
|---|---|---|---|---|
| Diameter | Thickness | Area mg/cm² | density | Porosity |
| (nm) | ((am) | area (mg/cm²) | (g/cm³) | (%) |
| 500 ± 120 | 30 ± 6 | 1.21 ± 0.13 | 0.43 ± 0.06 | 64-72 |

### Mechanical Property Characterization

Mechanical properties of the electrospun gelatin/TFE/PCL Tegaderm™ composite were measured using the tabletop uniaxial testing machine (INSTRON 3345). This was done using a 10-N load cell with a cross-head speed of 10 mm/min under ambient conditions. All samples were prepared in the form of rectangular shape with dimensions of 20×10 mm from the scaffold construct, with an average thickness of 120 μm measured from the digital micrometer. Four samples were tested for this characterization procedure. Specific sample preparation methodology is as mentioned in Z. M. Huang et al. [27]

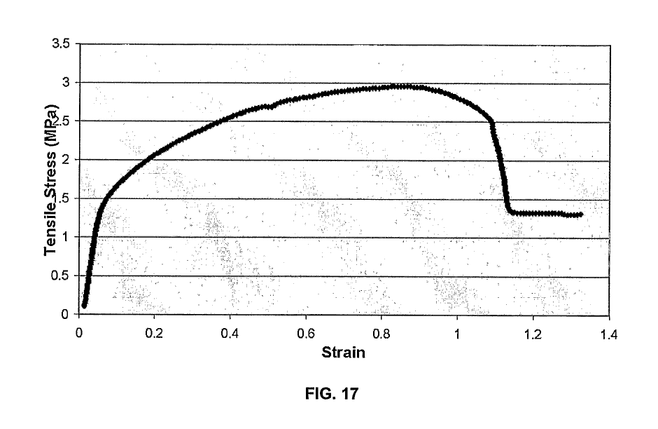

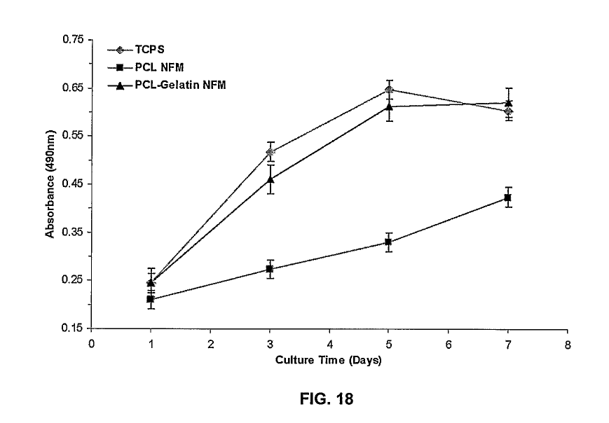

FIG. 16 shows the stress-strain behavior of the gelatin/TFE/PCL scaffold before and after detachment from the Tegaderm™ wound dressing. Interestingly, two different phases were noticed from the tensile loading graph. The first phase of the tensile loading graph is achieved with the gelatin/PCL scaffold still intact with the Tegaderm™ wound dressing. The second phase occurs after the scaffold has broken and tensile loading continues purely with Tegaderm™ wound dressing alone. This result has been extrapolated and shown in FIG. 17.

It is known that the combined polymer of gelatin/PCL has lower tensile and elongation properties Similarly, with an understanding that the Tegaderm™ wound dressing, has an almost elastic rubber-like tensile property, it is expected that the nanofibrous scaffold will break before the wound dressing does. However, the combined construct of the nanofibrous scaffold and Tegaderm™ wound dressing has given rise to an improvement in the tensile properties of a construct based solely on gelatin/PCL alone.

This Tegaderm-gelatin/PCL composite construct has shown to offer much better tensile strength, deformability and flexibility which is particularly important in skin rehabilitation procedures.

### Cell Viability Tests

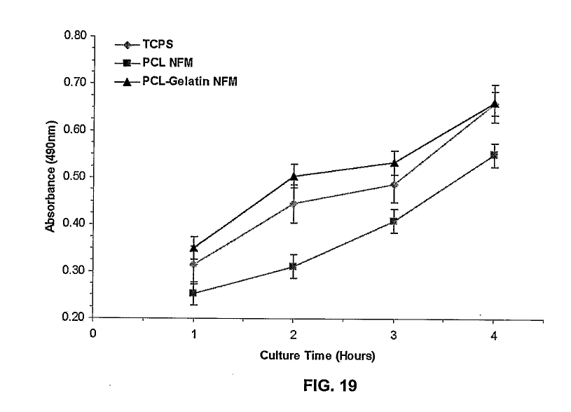

As shown from the optical density of cellular activities from the cell viability test from FIG. 18, it can be noticed that HDF cell viability increased greatly over the 7 days period on gelatin/PCL nanofiber scaffold as compared with the PCL scaffold. This result can be verified with cell counting test from FIG. 18. It is observed that cell counts on gelatin/PCL composite scaffold are approximately an 80% fold increase as compared with that of the normal PCL scaffold. It is interesting to note from FIG. 18 that by Day 7 of cell culture, optical density has begun to stagnate for the gelatin/PCL composite scaffold. This does not mean that cell growth has come to a stop, but rather at a slower rate. A probable reason will be at this phase the cells are working on penetrating into the composite scaffold through cell proliferation or growth in extracellularmatrix.

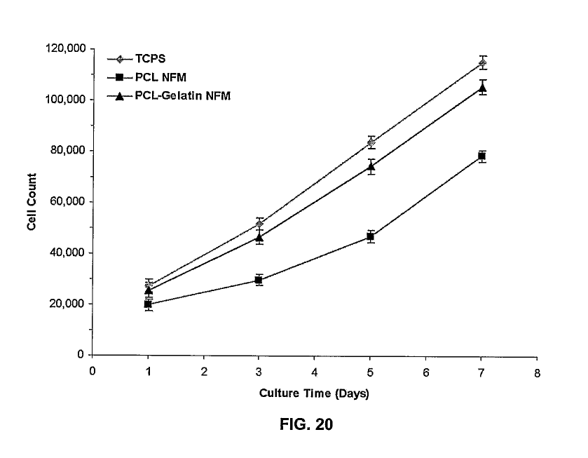

Results from FIG. 19 shows that the HDF cells, being anchorage cells, tend to attach better to the gelatin/PCL composite scaffold, achieving results that are almost comparable with that of TCPS. It can be concluded that the inclusion of the biopolymer gelatin into the PCL polymer solution has greatly enhanced the HDF affinity onto the scaffold structure.

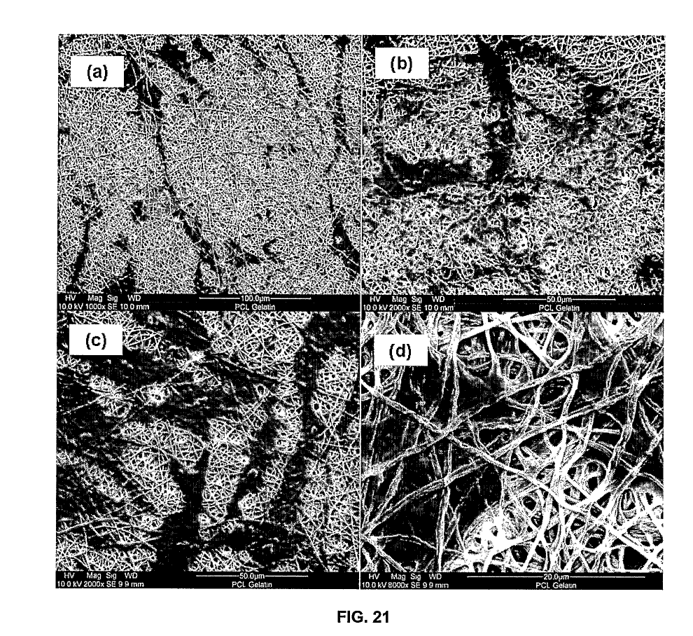

FIG. 20 shows the cell count of HDFs on TCPS, PCL NFM and PCL-Gelatin NFM. Cells were seeded at density of 1.5×10⁴ cells/well and cultured for a period of 7 days.

FIG. 20 shows FESEM morphological views of HDF proliferation on gelatin/PCL composite scaffold: (a) Day 1, (b) Day 3, (c) Day 5, (d) Initial HDF penetration into scaffold structure.

### Applications

It will be appreciated that the disclosed composite is highly useful in the treatment of dermal conditions such as skin burns. It will be appreciated that the skin cells seeded into the scaffold layer assists in the healing of the skin. Furthermore, the composite forms a protective barrier on the skin and prevents infection of the dermal, sub-dermal and epidermal tissue, particularly in situations where the body is unable to repair itself.

It will also be appreciated that the skin cells seeded into the scaffold layer provide a useful alternative, or compliment, to skin grafting. Additionally, as the inventors have surprisingly found that adhesives in commercially available polymer plasters, such as Tegaderm, do not kill or inhibit skin cell growth, the composite is able to be used in dermis and epidermis repair.

The composite additionally provides a useful alternative to the use of permanent skin replacement products. Furthermore, electrospinning the fibers onto the semi-permeable barrier provides a relatively low cost means to manufacture the composite. Furthermore, the composite can be used in ulcers that have signs of clinical infection or sinus tracts.

### REFERENCES

1. S. T. Boyce, G. D. Warden. Principles and practices for treatment of cutaneous wounds with cultured skin substitutes. The American Journal of Surgery. 2002; 183: 445-456.
2. K. G. Harding, H. L. Morris, G. K. Patel. Science, medicine and the future. Healing chronic wounds. Clinical Review. British Medical Journal. 2002; 324: 160-163.
3. J. L. Sorensen, B. Jorgensen, F. Gottrup. Surgical treatment of pressure ulcers. The American Journal of Surgery. July 2004; 188: 41S-51S.
4. B. L. Seals, T. C. Otero, A. Panitch. Polymeric biomaterials for tissue and organ regeneration. Materials Science and Engineering: R: Reports 34 (2001) 147-230.
5. Jennifer J. Marler, Joseph Upton, Robert Langer, Joseph P. Vacanti. Transplantation of cells in matrices for tissue regeneration. Advanced Drug Delivery Reviews 33 (1998) 165-182.
6. Stern R., McPherson M., Longaker M. Histologic study of artificial skin used in the treatment of full thickness thermal injury. Journal of Burn Care and Rehabilitation. (1990) 11: 7-13.
7. Integra Life Science: Integra® Dermal Regeneration Template—http://www.integra-1s.com/bus-skin_product.shtml
8. Smith & Nephew US—DERMAGRAFT* Diabetes/Tissue Engineering—http://www.dermagraft.com
9. Gentzkow G. D, Iwasaki S. D, Hershon K. S. Use of Dermagraft, a cultured human dermis, to treat diabetic foot ulcers. Diabetes Care. (1996) 19: 350-4.
10. Smith & Nephew US—TRANSCYTE* Biotechnology—http://wound.smith-nephew.com
11. Noordenbos J, Dore C, Hansbrough J. F. Safety and efficacy of Trancyte for the treatment of partial-thickness burns. Journal of Burn Care and Rehabilitation. (1999) 20: 245-281.
12. Using Skin Replacement Products to Treat Burns and Wounds—http://www.nursingcenter.com
13. A. Tay, T T Phan, P. See, C. Song, S T Lee. Cultured sub-confluent keratinocytes on wound dressing polymers for the treatment of burns and wounds. Wounds: A Compendium of Clinical Research and Practice 2000; 12 (5): 127-133.
14. T T Phan, I J Lim, E K Tan, A. Chua, B H Bay, S T Lee. Evaluation of cell culture on polyurethane-based membrane (TEGADERM™): Implication for tissue engineering of skin.
15. Luu Y. K., Kim K., Hsiao B. S., Chu B., Hadijiargyrou M. Development of a nanostructured DNA delivery scaffold via electrospinning of PLGA and PLA-PEG block copolymer. Journal of Controlled Release 89 (2003) 341-353.
16. Jamil A. Matthews, Gary E. Wnek, David G. Simpson, Gary L. Bowlin. Electrospinning of collagen nanofibers. Biomacromolecules 2002, 3, 232-238.
17. Chi H. Lee, Anuj Singla, Yugyung Lee. Biomedical applications of collagen. International Journal of Pharmaceutics 221 (2001) 1-22.
18. Gary E. Wnek, Marcus E. Carr, David G. Simpson, Gary L. Bowlin. Electrospinning of nanofiber fibrinogen structures. Nano Letters 2003, Vol. 3 No. 2, 213-216.
19. H. L Khor, K. W Ng, J. T Schantz, Toan-Thang Phan, T. C Lim, S. H Teoh, D. W Hutmacher. Poly (ε-caprolactone) films as a potential substrate for tissue engineering an epidermal equivalent. Materials Science and Engineering C 20 (2002) 71-75.
20. K. W Ng, D. W Hutmacher, J. T Schantz, C. S Ng, H. P Too, T. C Lim, T. T Phan, S. H Teoh. Evaluation of ultra-thin poly (ε-caprolactone) films for tissue engineered skin. Tissue Engineering Volume 7, Number 4, 2001. Mary Ann Liebert, Inc.
21. Wan-Ju Li, Cato T. Laurencin, Edward J. Caterson, Rocky S. Tuan, Frank K. Ko. Electrospun nanofibrous structure: A novel scaffold for tissue engineering. Journal of Biomedical Materials Research, Vol. 60, Issue 4, 613-621. (2002).
22. T. M. Freyman, I. V. Yannas, L. J. Gibson. Cellular materials as porous scaffolds for tissue engineering. Progress in Materials Science 46 (2001) 273-282.
23. Edwards C, Marks R. Evaluation of biomechanical properties of human skin. Clin Dermatol 1995; 13: 375-380.
24. Xu C. Y., Inai R., Kotaki M., Ramakrishna S. Aligned biodegradable nanofibrous structure: a potential scaffold for blood vessel engineering. Biomaterials 25 (2004) 877-886.
25. Yoshitomo H., Shin Y. M., Terai H., Vacanti J. P. A biodegradable nanofiber scaffold by electrospinning and its potential for bone tissue engineering. Biomaterials 24 (2003) 2007-2082.
26. Yanzhong Zhang, Hongwei Ouyang, Chwee Teck Lim, Seeram Ramakrishna, Zheng-Ming Huang. Electrospinning of Gelatin Fibers and Gelatin/PCL Composite Fibrous Scaffolds. J. Biomedical Materials Research Part B: Applied Biomaterials 72B: 156-165, 2005.
27. Zheng-Ming Huang, Y. Z. Zhang, S. Ramakrishna and C. T. Lim. Electrospinning and mechanical characterization of gelatin nanofibers. Polymer, Volume 45, Issue 15, 12 Jul. 2004, Pages 5361-5368
28. Zuwei Ma, Masaya Kotaki, Thomas Yong, Wei He and Seeram Ramakrishna. Surface engineering of electrospun polyethylene terephthalate (PET) nanofibers towards development of a new material for blood vessel engineering. Biomaterials, Volume 26, Issue 15, May 2005, Pages 2527-2536.
29. Wei He, ZuWei Ma, Thomas Yong, Wee Eong Teo and Seeram Ramakrishna. Fabrication of collagen-coated biodegradable polymer nanofiber mesh and its potential for endothelial cells growth. Biomaterials, Volume 26, Issue 36, December 2005, Pages 7606-7615.
30. S-H. Tan, R. Inai, M. Kotaki and S. Ramakrishna. Systematic parameter study for ultra-fine fiber fabrication via electrospinning process. Polymer, Volume 46, Issue 16, 25 Jul. 2005, Pages 6128-6134.
31. Anand S. Badami, Michelle R. Kreke, M. Shane Thompson, Judy S. Riffle and Aaron S. Goldstein. Effect of fiber diameter on spreading, proliferation, and differentiation of osteoblastic cells on electrospun poly(lactic acid) substrates. Biomaterials, In Press, Corrected Proof, Available online 15 Jul. 2005.
32. Melvin Schindler, Ijaz Ahmed, Jabeen Kamal, Alam Nur-E-Kamal, Timothy H. Grafe, H. Young Chung and Sally Meiners. A synthetic nanofibrillar matrix promotes in vivo-like organization and morphogenesis for cells in culture. Biomaterials, Volume 26, Issue 28, October 2005, Pages 5624-5631.
33. T. T. Phan, I. J. Lim, E. K. Tan, B. H. Bay & S. T. Lee, Evaluation of cell culture on the polyurethane-based membrane (Tegaderm™): implication for tissue engineering of skin, Cell & Tissue Engineering (2005) (T. T. Phan et al).

1. A composite comprising:
a semi-permeable barrier layer that is permeable to oxygen and impermeable to microorganisms; and
a scaffold fiber layer formed by electrospinning fibers on one side of said semi-permeable barrier layer.

2. A composite as claimed in claim 1, wherein at least one cell is provided within said scaffold fiber layer.

3. A composite as claimed in claim 2, wherein said at least one cell is a skin cell.

4. A composite as claimed in claim 3, wherein said skin cell is a human dermal fibroblast cell.

5. A composite as claimed in claim 1, wherein at least one cell is provided on the semi-permeable barrier layer.

6. A composite as claimed in claim 1, wherein the semi-permeable barrier layer is composed of a polymer.

7. A composite as claimed in claim 6, wherein the polymer is selected from the group consisting of polycellulose, polyurethane, polystyrene, polyimides, polyamides, resins, nylon, polysilicon, polyester, polyolefin, polyamide, polysilicone, copolymers and mixtures thereof.

8. A composite as claimed in claim 1, wherein the semi-permeable barrier layer is composed of at least one material selected from the group consisting of Tegaderm™, Dermagraft™, Transcyte™, Integra™ and Biobrane™.

9. A composite as claimed in claim 1, wherein the semi-permeable material is removable from said scaffold fiber layer.

10. A composite as claimed in claim 1, wherein an adhesive is provided on the semi-permeable barrier layer for allowing attachment to the skin of an animal or human.

11. A composite as claimed in claim 10, wherein the adhesive is selected from the group consisting of gelatin adhesives, resin based adhesives, phenol based adhesives, aldehyde based adhesives and mixtures thereof.

12. A composite as claimed in claim 1, wherein the average pore size of the semi-permeable barrier layer is in a range selected from the group consisting of 1 μm to 50 μm, 2 μm to 40 μm, 3 μm to 30 μm, 4 μm to 20 μm and 5 μm to 10 μm.

13. A composite as claimed in claim 1, wherein the scaffold fiber layer is comprised of a material selected from the group consisting of collagen, gelatin, keratin, chitosan, polypeptides, proteins, poly-ε-caprolactone (PCL), polyethylene oxide, polyvinyl alcohol, polyvinyl pyrolidon, polyamide, polylactic acid and mixtures thereof.

14. A composite as claimed in claim 1, wherein the fibers of the scaffold fiber layer are coaxial fibers.

15. A composite as claimed in claim 1, wherein the fiber size of the scaffold fiber layer are at least one of micro-sized fibers and nano-sized fibers.

16. A composite as claimed in claim 1, wherein the thickness of the scaffold fiber layer is selected from the group consisting of about 0.05 mm to about 5 mm, about 0.05 mm to about 4 mm, about 0.05 mm to about 3 mm, about 0.05 mm to about 2 mm, about 0.05 mm to about 1.5 mm, about 0.08 mm to about 1.5 mm, about 0.1 mm to about 1.5 mm, about 0.2 mm to about 1.5 mm, about 0.5 mm to about 1.5 mm, about 0.8 mm to about 1.5 mm.

17. A composite as claimed in claim 2, wherein said at least one cell is selected from the group consisting of embryonic stem cells, embryonic germ stem cells, fetal tissue derived epithelial cells, mesenchymal cells, endothelial stem/progenitor cells, bone marrow derived mesenchymal stem/progenitor cell, umbilical cord blood derived mesenchymal stem/progenitor cells, adipose tissue derived mesenchymal stem/progenitor cells, hair follicular epidermal stem cells, limbal epithelial stem cells, limbal epithelial stem cells, nail bed germ cells, osteoblast cells, chondrocytes, smooth muscle cells, tenocytes, buccal and oral mucosa keratinocytes and fibroblast cells, ligament fibroblast cells and periodental ligament fibroblasts cells.

18. A composite as claimed in claim 1, wherein the scaffold fiber layer comprises gelatin.

19. A method of making a composite comprising:
electrospinning fibers on a semi-permeable barrier layer that is permeable to oxygen and impermeable to microorganisms.

20. A method as claimed in claim 19, wherein the electrospinning comprises using a polymer melt or a polymer in solution, wherein the concentration of the polymer is sufficiently high to form a fiber during said electrospinning.

21. A method as claimed in claim 20, wherein the polymer melt or a polymer in solution is selected from the group consisting of collagen, gelatin, keratin, chitosan, polypeptides, proteins, poly-ε-caprolactone (PCL), polyethylene oxide, polyvinyl alcohol, polyvinyl pyrolidon, polyamide, polylactic acid and mixtures thereof

22. A method as claimed in claim 20, wherein the solvent of the solution is an organic solvent.

23. A method as claimed in claim 22, wherein the organic solvent is selected from the group consisting of alcohols, ketones, aldeydes and alkyl halides.

24. A method as claimed in claim 19, wherein said electrospinning comprises:
applying an electric field having a strength in the range selected from the group consisting of 5 kV to 25 kV, 5 kV to 20 kV, 5 kV to 15 kV, 5 kV to 10 kV, 6 kV to 15 kV, 6 kV to 14 kV and 8 kv to 12 kv.

25. A method as claimed in claim 19, wherein said electrospinning comprises:
applying an electric field having a length in the range selected from the group consisting of about 5 cm to about 25 cm, about 5 cm to about 20 cm, about 5 cm to about 15 cm, about 5 cm to about 10 cm, about 5 cm to about 25 cm, about 10 cm to about 25 cm and about 10 cm to about 15 cm.

26. A method as claimed in claim 19, wherein said electrospinning comprises:
using a syringe needle for forming the fibers.

27. A method as claimed in claim 26, comprising:
selecting said syringe having a radius in the range selected from the group consisting of about 0.1 mm to about 2 mm, about 0.1 mm to about 1 mm, about 0.1 mm to about 0.5 mm, about 0.1 mm to about 0.3 mm, about 0.2 mm to about 2 mm, and about 0.2 mm to about 1.2 mm.

28. A method as claimed in claim 27, comprising:
selecting said semi-permeable barrier layer that is permeable to oxygen and substantially impermeable to microorganisms.

29. An adhesive patch comprising:
a semi-permeable barrier layer that is permeable to oxygen and impermeable to microorganisms;
a scaffold fiber layer formed by electrospinning fibers on one side of said semi-permeable barrier layer; and
skin cells provided within said scaffold fiber layer.

30. A composite comprising:
a semi-permeable barrier layer;
at least two scaffold fiber layers, said scaffold fiber layers formed by electrospinning fibers on one side of said semi-permeable barrier layer; and
at least one cell provided in each of said at least two scaffold fiber layers, wherein said scaffold fiber layers comprise different cell types or the same cell types.

31. A method of making a composite comprising:
electrospinning fibers on a semi-permeable barrier layer to form a scaffold fiber layer; and
seeding at least one cell in said scaffold fiber layer.

32. Use of an adhesive patch according to claim 29 for treating the skin condition of an animal.

33. Use of an adhesive patch according to claim 32, wherein said skin condition is a burn.

34. A kit for treating a dermal condition of an animal comprising an adhesive patch of claim 29 together with instructions for applying the adhesive patch to the skin of an animal having said dermal condition.

35. A method for treating a skin condition of an animal comprising applying an adhesive patch of claim 29 to the skin of the animal.

36. A method as claimed in claim 35, wherein said skin condition is a dermal burn.

37. A polyurethane membrane comprising one or more cells growing thereon.

38. A polyurethane membrane as claimed in claim 37, wherein said polyurethane membrane is Tegaderm™.
